(19) United States
(12) Patent Application Publication
(10) Pub. No.: US 2017/0347685 A1
(43) Pub. Date: Dec. 7, 2017

(54) HORSE SUPPLEMENT CARRIER AND FEEDING PRODUCT

(71) Applicant: Nancy Lee POHLMAN, CAMP VERDE, AZ (US)

(72) Inventor: Nancy Lee POHLMAN, CAMP VERDE, AZ (US)

(21) Appl. No.: 15/170,732

(22) Filed: Jun. 1, 2016

(51) Int. Cl. A23K 50/20; A61K 47/22; A61K 9/00; A23K 10/33; A23K 10/30; A61K 47/46; A23K 40/20

(52) U.S. Cl. CPC A23K 50/20; A61K 47/46; A61K 47/22; A23K 40/20; A23K 10/33; A23K 10/30; A61K 9/009

(57) ABSTRACT

Some embodiments of the present disclosure include a horse carrier and delivery product for administering a pill or supplement paste or liquid to an animal, such as a horse. The carrier and delivery system may include a shell having a pocket; and, optionally, a paste filling the pocket, the paste being configured to accept a pill and secure the pill within the pocket. The shell may be made of a mixture of beet pulp, horse feed, white flour, fenugreek, ascorbic acid, fruit pectin, alfalfa greens, water, vitamin E oil, and sea salt. The paste may be made of a mixture of raisins, molasses, and Vitamin E oil. In embodiments, the shell and the paste may be configured to mask a flavor of the pill placed therein.

### BACKGROUND

The embodiments herein relate generally to animals, and more particularly, to a pill or carrier and feeding product for a horse.

Cushing's Disease (PPID) is a disease that affects a number of horses. A horse must be given a Prascend pill every day for control of clinical signs associated with this disease. However, many pills, including Prascend pills, given to horses are rejected by the animal due to poor taste or the lack of palatability. Trying to deliver a pill to a horse in its feed is a hit or miss proposition without the assurance that the pill was actually delivered to the horse.

Moreover, some supplements are delivered to a horse in a liquid or paste form. However, these can also be difficult to administer to a horse or other animal.

Therefore, what is needed is a pill and paste carrier and feeding product configured to deliver a pill or paste supplement to a horse and also mask the taste of the supplement, while also being formulated with low carbohydrate and sugar content.

### SUMMARY

Some embodiments of the present disclosure include a horse pill or paste carrier and delivery product. The carrier and delivery system may include a shell having a pocket; and, optionally, a paste filling the pocket, the paste being configured to accept a pill and secure the pill within the pocket. The shell may be made of a mixture of beet pulp, horse feed, white flour, fenugreek, ascorbic acid, fruit pectin, alfalfa greens, water, vitamin E oil, and sea salt. The paste may be made of a mixture of raisins, molasses, and Vitamin E oil. In embodiments, the shell and the paste may be configured to mask a flavor of the pill placed therein

### BRIEF DESCRIPTION OF THE FIGURES

The detailed description of some embodiments of the invention is made below with reference to the accompanying figures, wherein like numerals represent corresponding parts of the figures.

FIG. 1 is a perspective view of one embodiment of the present disclosure.

FIG. 2 is a cross-sectional view of one embodiment of the present disclosure.

FIG. 3 is a top perspective view of one embodiment of the present disclosure.

FIG. 4 is a perspective view of one embodiment of the present disclosure.

FIG. 5 is a cross-sectional view of one embodiment of the present disclosure.

### DETAILED DESCRIPTION OF CERTAIN EMBODIMENTS

In the following detailed description of the invention, numerous details, examples, and embodiments of the invention are described. However, it will be clear and apparent to one skilled in the art that the invention is not limited to the embodiments set forth and that the invention can be adapted for any of several applications.

The device of the present disclosure may be used to deliver a pill to a horse and may comprise the following elements. This list of possible constituent elements is intended to be exemplary only, and it is not intended that this list be used to limit the device of the present application to just these elements. Persons having ordinary skill in the art relevant to the present disclosure may understand there to be equivalent elements that may be substituted within the present disclosure without changing the essential function or operation of the device.

1. Shell

2. Optional Paste

The various elements of the device of the present disclosure may be related in the following exemplary fashion. It is not intended to limit the scope or nature of the relationships between the various elements and the following examples are presented as illustrative examples only.

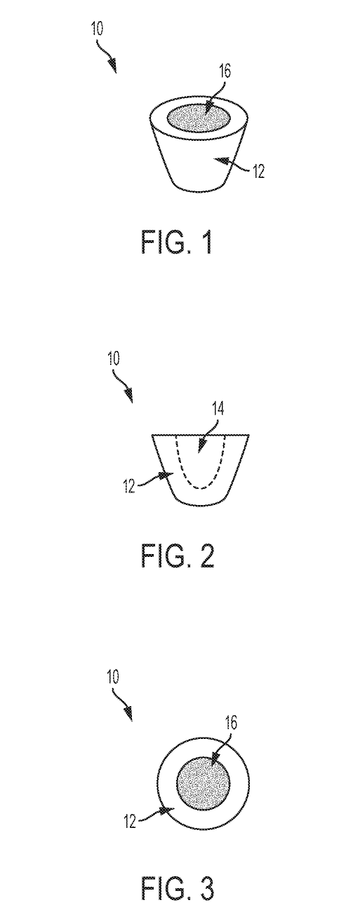

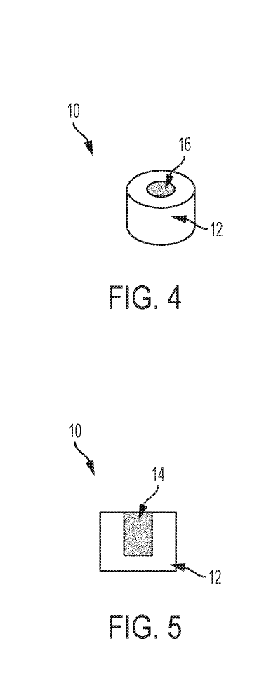

By way of example, and referring to FIGS. 1-5, some embodiments of the present disclosure include a horse pill pocket 10 comprising an outer shell 12 defining an inner pocket 14 and, optionally, a paste 16 configured to fill the pocket 14, the paste 16 also configured to secure a pill within the inner pocket 14, wherein the shell 12 and the paste 16 comprise ingredients that are palatable to horses and that mask the taste of an equine pill, such as Prascend Tablets, which are administered to treat Cushing Disease (PPID). Alternatively, the inner pocket 14 may be configured to accommodate a supplement paste or liquid, such as bimectin (which may remove worms and bots), zimecterin (which may be a paste dewormer), strongid paste (which may remove internal parasites), perfect balance electrolyte paste, OTC jug, (a vitamin and mineral paste), micro B-1 paste, oral X-B (a mineral complex for iron and copper deficiency), micro B-12 5X (a vitamin B-12 paste), and the like. In such embodiments, the carrier may not include the paste 16 and instead may include only the supplement paste or liquid.

The shell 12 may comprise any desired ingredients and, in some embodiments, comprises a mixture of beet pulp, horse feed, safflower oil, vitamin E oil, alfalfa greens, fenugreek, ascorbic acid (vitamin C), wheat, oats, peppermint oil, water, sea salt, fruit pectin (or apple pectin). However, some embodiments may not comprise any oats. The shell 12 may further comprise mineral oil and flour, which may act as binding agents for the mixture. Some embodiments may also comprise peppermint oil. The shell 12 ingredients may be mixed into a moldable paste, which may be deposited in a mold, forming a cylinder, such as a cylinder having a diameter of about 1 inch and a depth of about 1 inch. The pocket 14 may then be made in each molded item. For example, a pocket 14 having a diameter of 1¼ inch and a depth of about ½ inch may be made in the shell 12. The shell 12 may be allowed to dry and removed from the mold, creating a hard, crunchy shell 12 having a pocket 14. While the above sizes are described, any size shell 12 and pocket 14 may be formed, as needed or desired.

The paste 16 may comprise any desirable ingredients and, in some embodiments, comprises a mixture of raisins, molasses, and safflower or Vitamin E oil. The paste 16 may have a thick, sticky consistency and, thus, may be softer than the shell 12. The paste 16 may then be used to substantially fill the pocket 14.

In embodiments, either the shell 12 or the paste 16 may further comprise a flavoring, such as fenugreek (maple), peppermint, banana, cherry, mint, or any other desired flavoring.

### Example 1: Making the Shell

About four cups of ground beet pulp were mixed with about 4 cups of water. The combination was mixed well and allowed to seat for about 5 minutes. Meanwhile, about 8 cups of horse feed and about 4 cups of water were mixed. About ¾ cup of safflower oil (Vitamin E oil) was added to the horse feed/water mixture. The mixture was mixed well and allowed to sit for about 5 minutes. Meanwhile, about 25 cups of ground alfalfa, about 8 cups of white flour, about 1 cup of sea salt, about 1½ cups powdered fenugreek, about ¾ cups powdered ascorbic acid (vitamin C), and about ¾ cups powdered fruit pectin or apple pectin were mixed together. All of the dry ingredients were thoroughly blended together. The wet beet pulp and the wet horse feed were added to the dry ingredients and mixed well. Approximately 40 oz. of water was blended into the batch a little at a time. A slow steady stream of water or spraying the water into the batch while it is being mixed may work best. This process may create the moldable paste, which may be formed into the shell.

### Example 2: Making the Paste

About 2 cups of raisins, about ½ cup of molasses, and about 2 tablespoons of safflower oil (vitamin E oil) may be added to a blender and blended until smooth, resulting in the paste.

In embodiments, a mixture of safflower and vitamin E may be used. For example, 6 parts of safflower oil to 1 part of pure vitamin E oil may be mixed, creating the mixture. Vitamin E oil is very thick and sticky and tends to be difficult to use. It may flow better when mixed with safflower oil.

To use the horse pill carrier 10, a user may insert a pill into the paste 16 and feed the pill carrier 10 to a horse. Because of the ingredients used, the taste of a pill may be masked, resulting in the horse eating the pill and pill carrier 10 without the horse rejecting the pill. Alternatively, in the case of a paste carrier, a paste or liquid supplement may be inserted into the shell and the product may be fed to an animal, such as a horse.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A horse carrier and delivery product for administering a supplement to a horse, the carrier and delivery product comprising:
a shell having a pocket; and
optionally, a paste filling the pocket, the paste being configured to accept a pill and secure the pill within the pocket,
wherein:
the shell comprises a mixture of beet pulp, horse feed, white flour, fenugreek, ascorbic acid, fruit pectin, alfalfa greens, water, vitamin E oil, and sea salt;
the paste comprises a mixture of raisins, molasses, and Vitamin E oil; and
the shell and the paste are configured to mask a flavor of the pill placed therein.

2. The horse carrier and delivery product of claim 1, wherein a consistency of the paste is softer than a consistency of the shell.

3. The horse carrier and delivery product of claim 1, wherein the shell further comprises at least one member selected from the group consisting of oats, mineral oil, wheat, and peppermint oil.

4. The horse carrier and delivery product of claim 1, wherein:
the shell further comprises safflower oil; and
a ratio of safflower oil to vitamin E oil is about 6:1.

5. The horse carrier and delivery product of claim 1, wherein:
the paste further comprises safflower oil; and
a ratio of safflower oil to vitamin E oil is about 6:1.

6. The horse carrier and delivery product of claim 1, wherein the fruit pectin is apple pectin.

7. The horse carrier and delivery product of claim 1, wherein:
a batch of the shell comprises:
about 4 cups ground beet pulp;
about 13 cups water;
about 8 cups horse deed;
about ¾ cups of a safflower oil/vitamin E oil mixture;
about 25 cups of ground alfalfa;
about 8 cups of white flour;
about 1 cup sea salt;
about 1½ cups powdered fenugreek;
about ¾ cups powdered ascorbic acid; and
about ¾ cup powdered fruit pectin; and
a batch of the paste comprises:
about 2 cups raisins;
about ½ cup molasses; and
about 2 tablespoons of the safflower oil/vitamin E oil mixture.
